(19) United States
(12) Patent Application Publication
(10) Pub. No.: US 2024/0084377 A1
(43) Pub. Date: Mar. 14, 2024

(54) METHODS FOR THE SEQUENCING OF PHOSPHORODIAMIDATE MORPHOLINO OLIGOMERS (PMO) AND PEPTIDE-PMO (PPMO) CONJUGATES

(71) Applicant: Entrada Therapeutics, Inc., Boston, MA (US)

(72) Inventor: Sergey Shuvaev, Quincy, MA (US)

(21) Appl. No.: 18/238,725

(22) Filed: Aug. 28, 2023

Related U.S. Application Data
(60) Provisional application No. 63/373,968, filed on Aug. 30, 2022.

(51) Int. Cl. C12Q 1/6869

(52) U.S. Cl. CPC C12Q 1/6869

(57) ABSTRACT

The disclosure relates to a method for sequencing a biotinylated phosphorodiamidate morpholino oligomer (PMO).

### BACKGROUND

Phosphorodiamidate morpholino oligomers (PMO) are single-stranded DNA analogs that are built upon a backbone of morpholine rings connected by phosphorodiamidate linkages. PMOs and PPMOs are attractive as alternatives to DNA and RNA therapeutics because of their hydrolytic stability. For example, PMOs have been evaluated for resistance to a variety of enzymes (e.g., nucleases, proteases, esterases, and serum) and have been found to be completely resistant to 13 different hydrolases and serum and plasma. See Hudziak, et al., Antisense & Nucleic Acid Drug Development 6: 267-272 (1996), which is incorporated by reference as if fully set forth herein. The stability of PMOs stems at least from the stable phosphoramidate linkages indicated by the dashed line in Scheme I:

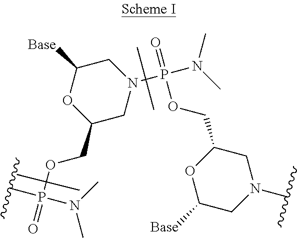

Although the excellent resistance of PMOs to enzymatic attack makes them attractive for in vivo use, that same feature makes PMOs difficult to digest, for example, to determine the sequence of the PMO. Further, the lack of the charge of PMOs precludes separation of digested fragments in an external electric field so that they can be sequenced.

### SUMMARY

Methods for the chemical sequencing of PMOs and PPMOs are therefore needed. The instant disclosure provides various examples of such methods.

### DESCRIPTION OF THE DRAWINGS

The drawings illustrate generally, by way of example, but not by way of limitation, various embodiments discussed herein.

FIG. 1 is a schematic of the digestion and purification methods described herein for PMOs.

FIG. 2 is a MALDI-TOF spectrum of PMO-1-PEG₂-biotin showing a ladder-like deletion pattern after purification. The PMO-1 sequence is 5′ AAA CGC CGC CAT TTC TCA ACA GAT C 3′.

FIG. 3 is a schematic of the streptavidin purification method described herein for PMOs.

FIG. 4A is a MALDI-TOF spectrum of PMO-1 using 2,5-dihydroxybenzoic acid as a matrix.

FIG. 4B is a MALDI-TOF spectrum of PPMO-1-EEV using α-cyano-4-hydroxycinnamic acid (B), wherein the generic structure of PPMO-1-EEV is shown in FIG. 5 without showing the 5′ AAA CGC CGC CAT TTC TCA ACA GAT C 3′ sequence for PMO-1 for the sake of simplicity.

FIG. 5 is the generic chemical structure of PMO-1-EEV.

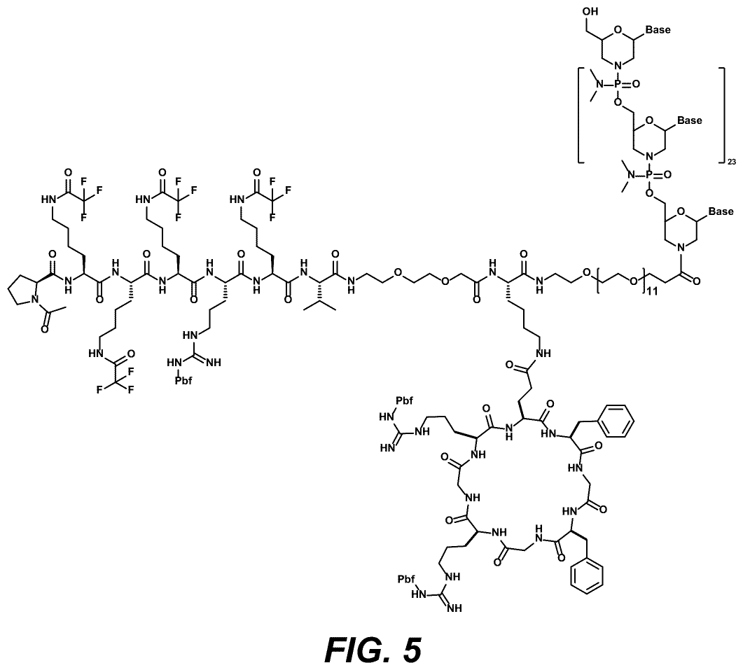

### DESCRIPTION

Reference will now be made in detail to certain embodiments of the disclosed subject matter. While the disclosed subject matter will be described in conjunction with the enumerated claims, it will be understood that the exemplified subject matter is not intended to limit the claims to the disclosed subject matter.

The disclosure relates to a method for sequencing a phosphorodiamidate morpholino oligomer (PMO), the method comprising:
(a) conjugating a PMO to a biotin moiety to form a biotinylated PMO;
(b) contacting the biotinylated PMO with an acid to obtain a crude digestion product;
(c) contacting the crude digestion product with streptavidin-coated magnetic beads to obtain a magnetic bead isolate comprising free PMO and a magnetic bead complex comprising biotinylated PMO and streptavidin-coated magnetic beads;
(d) washing the magnetic bead isolate to remove free PMO from the magnetic bead complex to obtain a purified magnetic bead isolate;
(e) releasing the streptavidin-coated magnetic beads from the magnetic bead complex to obtain a purified digestion product; and
(f) analyzing the purified digestion product.

As used herein, the terms “phosphorodiamidate morpholino oligomer” and “PMO” generally refer to compounds that are nucleotide analogs in which the ribose or deoxyribose sugar is replaced by a 6-membered morpholino ring, and the anionic phosphodiester linkage between adjacent morpholino rings is replaced by a non-ionic phosphorodiamidate linkage, as shown in Scheme II for example:

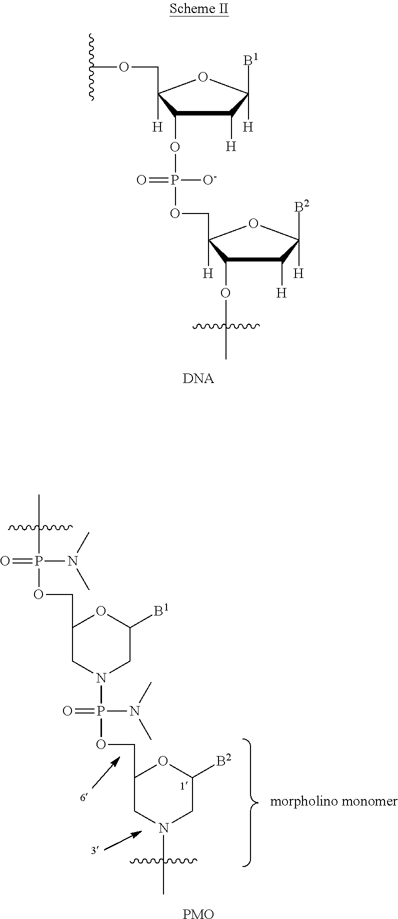

wherein B¹ and B² are the same or different and can a nitrogenous base such as adenine, thymine, cytosine, guanine, uracil, or derivatives thereof. Derivatives of nitrogenous bases include, for example, methylation of the bases and oxidation products of the bases. In the case of cytosine, for example, derivatives can include 5-methylcytosine and oxidation products thereof, including 5-hydroxymethylcytosine, 5-formylcytosine, and 5-carboxylcytosine. Derivatives of adenine include N⁶-methyladenine and N¹-methyladenine. Other nitrogenous base derivatives include inosine and pseudouridine. See, e.g., Front Genet. 9: 640 (2018) and Nature 541: 339-346 (2017), each of which is incorporated by reference as if fully set forth herein.

The PMOs contemplated herein can have the repeating sequence:

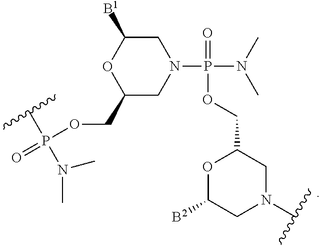

In addition, the PMOs contemplated herein include peptide-PMO (PPMO) conjugates. Examples of peptide-PMO conjugates include those of the formula:

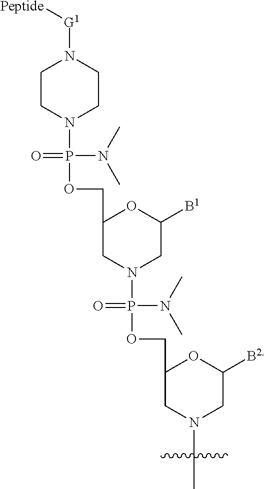

wherein G¹ can be absent or is a suitable linking group. In some examples, G¹ can comprise a β-alanyl group to which the peptide or polypeptide can be linked to the PMO. Thus, an example of a PPMO is:

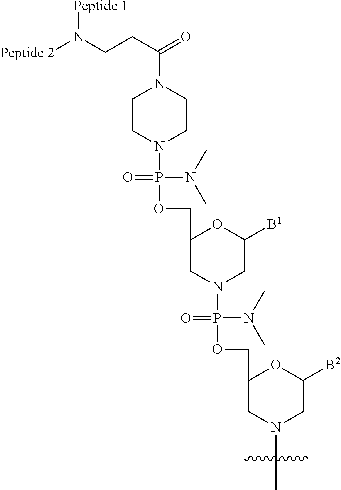

where Peptide 2 can be absent or Peptide 1 and Peptide 2, when present, can be the same or different. See, e.g., Biochimica et Biophysica Acta (BBA)—Biomembranes 1798: 2296-2303 (2010), which incorporated by reference as if fully set forth herein. Peptide 1 and/or Peptide 2 can be cell-penetrating peptides.

The PMOs contemplated herein can comprise any suitable number of morpholino monomers, for example, from 5 to 100 morpholino monomers (e.g., 5 to 50, 10 to 50, 15 to 30 or 20 to 30 morpholino monomers, or 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 morpholino monomers).

In a non-limiting example, the biotin moiety:

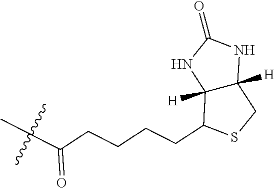 [SMILES: *C(=O)CCCCC1SC[C@H]2NC(=O)N[C@@H]12]

is conjugated to the 3′ end of the PMO, where the “3′ end” is shown in Scheme II. In one example, the PMO 3′-nitrogen can be conjugated directly to the biotin moiety (e.g., the PMO 3′-nitrogen can be conjugated to the biotin carbonyl shown above) to form an amide. In another example, the PMO is conjugated to the biotin moiety through a linker, such that the biotinylated PMO has the formula PMO-L-biotin, wherein L is a linker and the linker is covalently coupled to a 3′-end of the PMO. Thus, for example, the PMO-L-biotin is of the formula:

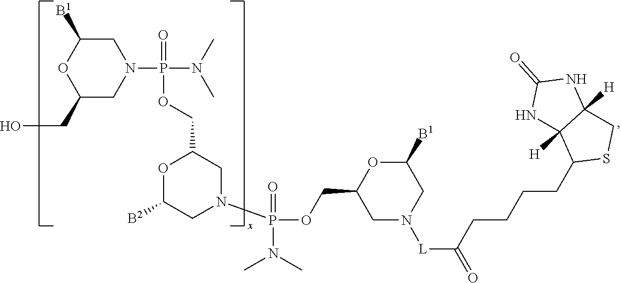

wherein each B¹ and B² is, independently, adenine, thymine, cytosine, guanine, uracil, or derivatives thereof; and x is an integer from 10 to 100 (e.g., 10 to 50, 20 to 100, 30 to 90, 50 to 100 or 30 to 100).

The linker, L, can be any suitable linker. Examples of suitable linkers include linkers comprising a divalent polyethylene glycol (PEG) group. The linker can comprise ionizable functional groups. Such groups can include a functional group that can be readily converted into an ionic functional group, such as an ionizable carboxy group (—CO₂H) that can be readily deprotonated to form a carboxylate anion (—CO₂⁻). As used herein, the terms “ionizable” and “ionic” are used interchangeably. The ionizable functional groups contemplated herein can be connected to the linker by way of one or more linking moieties. Furthermore, a single ionizable functional group can be extended from a single linking moiety, or a plurality of ionic moieties can have one or more linking moieties therebetween. For instance, the ionizable functional groups can have any of the following structures: -L^{A}-X^{A} or -L^{A}-(L^{A}′-X^{A})₂, in which each L^{A} and L^{A}′ is a linking moiety; and each X^{A} includes, independently, an acidic moiety, a basic moiety, or a multi-ionic moiety. Non-limiting linking moieties (e.g., for L^{A} and L^{A}′) include a covalent bond —O—, —NR^{N1}—, —SO₂—NR^{N1}-Ak-, —(O-Ak)_{L1}-SO₂—NR^{N1}-Ak-, -Ak-, -Ak-(O-Ak)_{L1}-, —(O-Ak)_{L1}-, -(Ak-O)_{L1}-, —C(O)O-Ak-, —Ar—, or —Ar—O—, in which Ak is an optionally substituted alkylene, R^{N1} is H or optionally substituted alkyl, Ar is an optionally substituted arylene, and L1 is an integer from 1 to 10 (e.g., 1 to 5 or 1 to 3). Thus, for example, L^{A} can be —(CH₂)_{L1}—, —O(CH₂)_{L1}—, —(CF₂)_{L1}—, —O(CF₂)_{L1}—, or —S(CF₂)_{L1}—, in which L1 is an integer from 1 to 10 (e.g., 1 to 5 or 1 to 3).

Non-limiting ionizable functional groups include carboxy (—CO₂H), carboxylate anion (—CO₂⁻), a guanidinium cation (e.g., —NR^{N1}—C(═NR^{N2}R^{N3})(NR^{N4}R^{N5}) or >N═C(NR^{N2}R^{N3}) (NR^{N4}R^{N5})), or a salt form thereof. Non-limiting examples of each of R^{N1}, R^{N2}, R^{N3}, R^{N4}, and R^{N5} is, independently, H, optionally substituted alkyl, optionally substituted aryl, or optionally substituted amino; or R^{N1} and R^{N2}, R^{N2} and R^{N3}, R^{N3} and R^{N4}, R^{N1} and R^{N2}, or R^{N1} and R^{N4} taken together with the nitrogen atom to which each are attached, form an optionally substituted heterocyclyl, heterocycle, or heterocyclic cation.

Ionizable functional groups can also include one or more sulfur atoms. Non-limiting sulfur-containing moieties include sulfo (—SO₂OH), sulfonate anion (—SO₂O⁻), sulfonium cation (e.g., —SR^{S1}R^{S2}), sulfate (e.g., —O—S(═O)₂(OR^{S1})), sulfate anion (—O—S(═O)₂O⁻), or a salt form thereof. Non-limiting examples of each of R^{S1} and R^{S2} is, independently, H, optionally substituted alkyl, optionally substituted aryl, or optionally substituted amino; or R^{S1} and R^{S2} taken together with the sulfur atom to which each are attached, form an optionally substituted heterocyclyl, heterocycle, or heterocyclic cation; or R^{S1} and R^{S2}, taken together, form an optionally substituted alkylene or heteroalkylene.

Ionizable functional groups can also include one or more phosphorous atoms. Non-limiting phosphorous-containing moieties include phosphono (e.g., —P(═O)(OH)₂), phosphonate anion (e.g., —P(═O)(O⁻)₂ or —P(═O)(OH)(O⁻)), phosphate (e.g., —O—P(═O)(OR^{P1})(OR^{P2}) or —O—[P(═O)(OR^{P1})—O]_{P3}—R^{P2}), phosphate anion (e.g., —O—P(═O)(OR^{P1})(O⁻) or —O—P(═O)(O⁻)₂), phosphonium cation (e.g., —P⁺R^{P1}R^{P2}R^{P3}), phosphazenium cation (e.g., —P⁺(═NR^{N1}R^{N2})R^{P1}R^{P2}, in which each of R^{N1} and R^{N2} is, independently, optionally substituted alkyl or optionally substituted aryl), or a salt form thereof. Non-limiting examples of each of R^{P1}, R^{P2}, and R^{P3} is, independently, H, optionally substituted alkyl, optionally substituted aryl, or optionally substituted amino; or R^{P1} and R^{P2}, taken together with the phosphorous atom to which each are attached, form an optionally substituted heterocyclyl, heterocycle, or heterocyclic cation; or R^{P1} and R^{P2} and R^{P3}, taken together with the phosphorous atom to which each are attached, form an optionally substituted heterocyclyl, heterocycle, or heterocyclic cation; or a single, double, or non-localized pi bond, provided that a combination of bonds result in a tetravalent phosphorous; or wherein two of R^{P1}, R^{P2}, and R^{P3}, taken together, form an optionally substituted alkylene or heteroalkylene.

Yet other ionizable functional groups can include one or more nitrogen atoms. Non-limiting nitrogen-containing moieties include amino (e.g., —NR^{N1}R^{N2}), ammonium cation (e.g., —N⁺R^{N1}R^{N2}R^{N3} or —N⁺R^{N1}R^{N2}—) heterocyclic cation (e.g., piperidinium, 1,1-dialkyl-piperidinium, pyrrolidinium, 1,1-dialkyl-pyrrolidinium, pyridinium, 1-alkylpyridinum, (1,4-diazabicyclo [2.2.2]octan-1-yl) (DABCO), 4-alkyl-(1,4-diazabicyclo[2.2.2]octan-1-yl), etc.), or a salt form thereof. Non-limiting examples of each of R^{N1}, R^{N2}, and R^{N3} is, independently, H, optionally substituted alkyl, optionally substituted cycloalkyl, or optionally substituted aryl; or R^{N1} and R^{N2}, taken together with the nitrogen atom to which each are attached, form an optionally substituted heterocyclyl, heterocycle, or heterocyclic cation; or R^{N1} and R^{N2} and R^{N3}, taken together with the nitrogen atom to which each are attached, form an optionally substituted heterocyclyl, heterocycle, or heterocyclic cation; or wherein two of R^{N1}, R^{N2}, and R^{N3}, taken together, form an optionally substituted alkylene or heteroalkylene; or a single, double, or non-localized pi bond, provided that a combination of bonds result in a tetravalent nitrogen.

Thus, for example, the linker, L, can comprise or can be a divalent group of the formula:

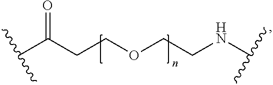

wherein n is an integer from 1 to 10 (e.g., 1, 2, 3, 4, 5, 6, 7, 8, 9, 10, 2 to 5, 2 to 6, 1 to 8, 5 to 10 or 4 to 9). For example, the linker can comprise a divalent group of the formula:

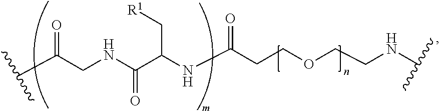

wherein:
R¹ comprises an ionizable group;
m is an integer from 1 to 10 (e.g., 1, 2, 3, 4, 5, 6, 7, 8, 9, 10, 2 to 5, 2 to 6, 1 to 8, 5 to 10 or 4 to 9); and
n is an integer from 1 to 10 (e.g., 1, 2, 3, 4, 5, 6, 7, 8, 9, 10, 2 to 5, 2 to 6, 1 to 8, 5 to 10 or 4 to 9).

In one example, the linker can comprise a divalent group of the formula:

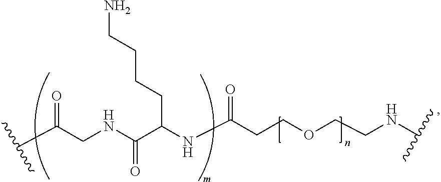

wherein:
m is an integer from 1 to 10 (e.g., 1, 2, 3, 4, 5, 6, 7, 8, 9, 10, 2 to 5, 2 to 6, 1 to 8, 5 to 10 or 4 to 9); and
n is an integer from 1 to 10 (e.g., 1, 2, 3, 4, 5, 6, 7, 8, 9, 10, 2 to 5, 2 to 6, 1 to 8, 5 to 10 or 4 to 9).

In one example, the -L-biotin group can comprise or is a group of the formula:

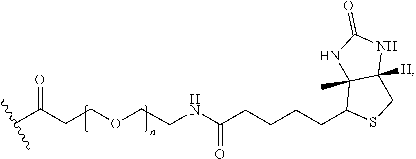

wherein n is an integer from 1 to 10 (e.g., 1, 2, 3, 4, 5, 6, 7, 8, 9, 10, or 2 to 5, 2 to 6, 1 to 8, 5 to 10 or 4 to 9). When n is 2, the PEG portion of L is called “PEG2” or “PEG₂,” which are used interchangeably herein.

In any of the foregoing examples, m is 2, n is 2 or both m and n are each 2.

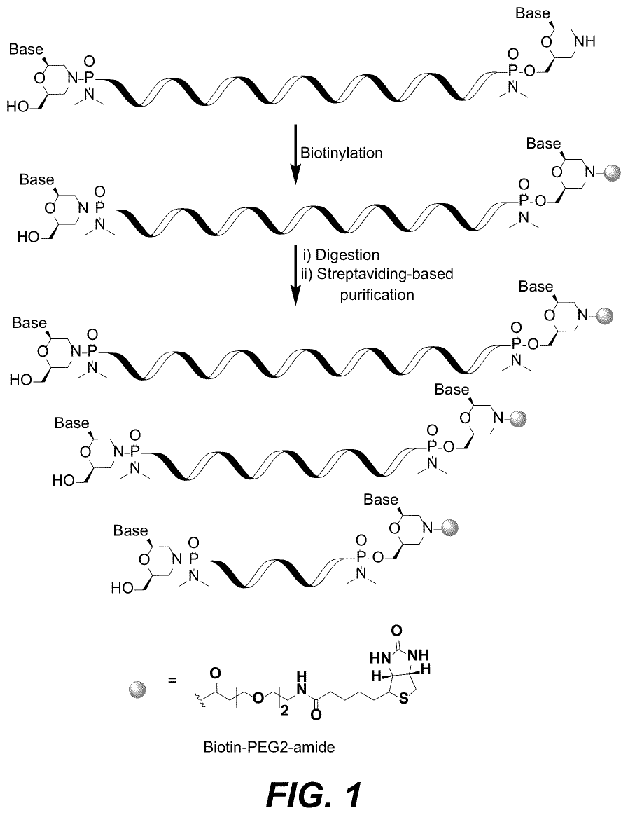

The methods for sequencing a PMO described herein include conjugating a PMO to a biotin moiety to form a biotinylated PMO. An example of the conjugating is shown in FIG. 1, wherein, for example, the carboxylic acid of a compound of the formula:

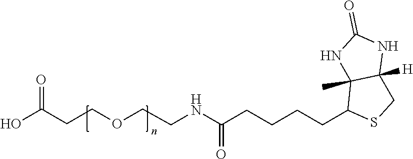

is conjugated with the terminal morpholino nitrogen of a compound of the formula:

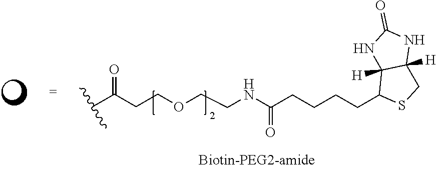

wherein:

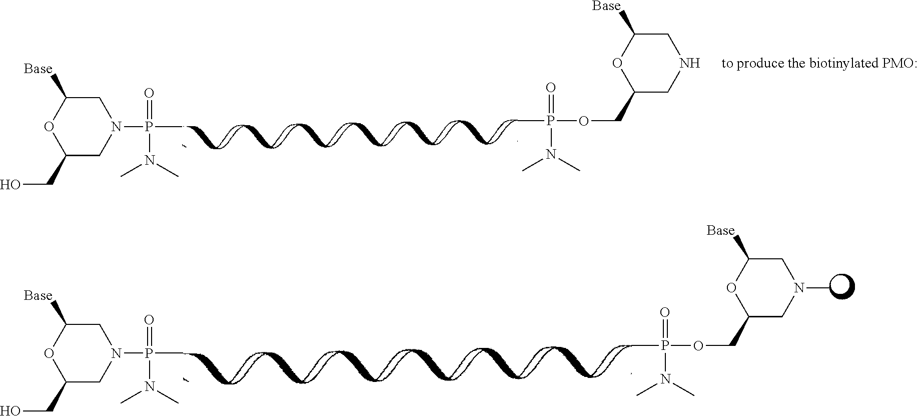

The methods for sequencing a PMO described herein include contacting the biotinylated PMO with an acid to obtain a crude digestion product. While not wishing to be bound by any specific theory, the contact of the PMO with the acid is believed to cause the hydrolysis of the phosphoroamidate bond as shown in Scheme III:

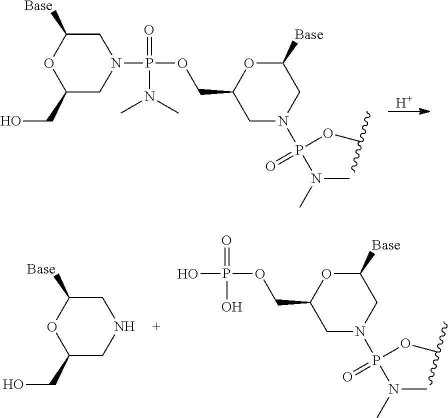

The “contacting” can comprise mixing the biotinylated PMO with the acid in a suitable container as described herein. Suitable acids that can be used in the methods described herein include at least one inorganic acid. Examples of inorganic acids that can be used include hydrochloric acid, sulfuric acid, trifluoroacetic acid (TFA) or the like. Thus, for example, an aqueous solution of biotinylated PMO (e.g., a solution of the biotinylated PMO in deionized water) can be contacted with an aliquot of acid (e.g., 100 μL of 0.3 M solution of TFA in DI water) at a suitable temperature (e.g., 37° C.) for a suitable amount of time (e.g., from 30 minutes to 1 hour). The resulting solution can then be allowed to cool (e.g., to room temperature, “rt”) and the acid can be neutralized. The resulting solution can then be lyophilized.

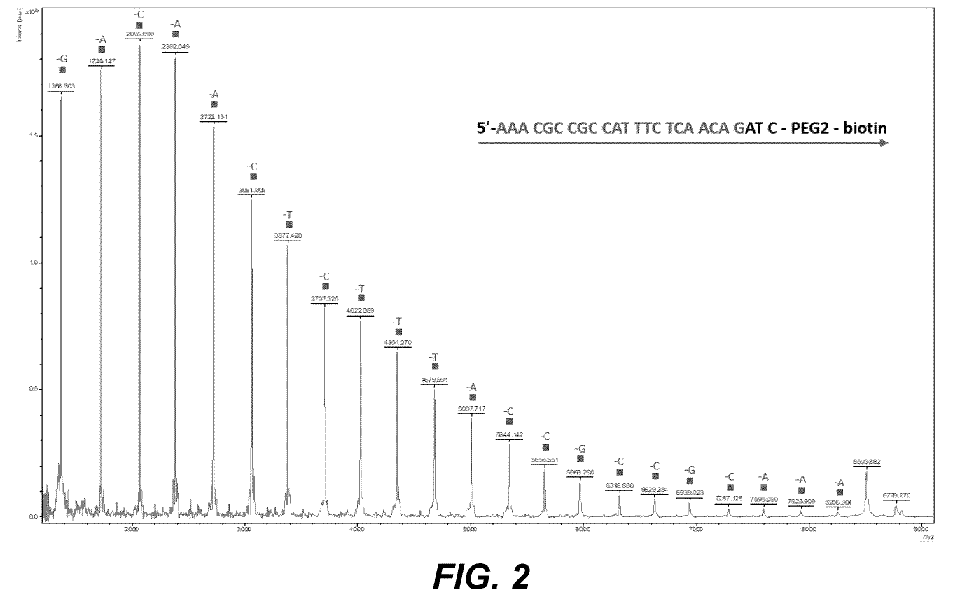

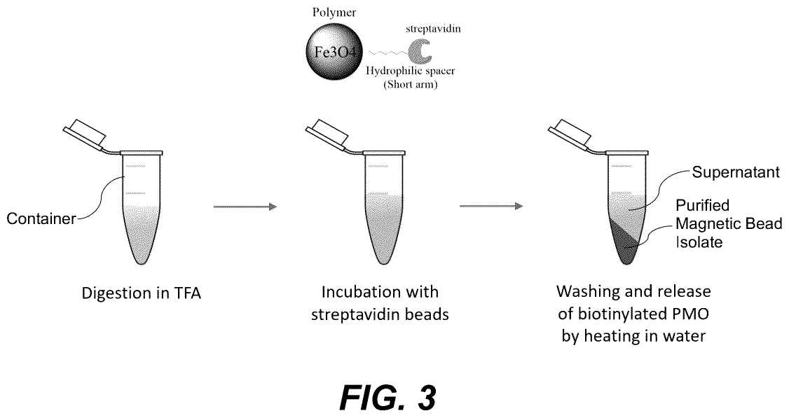

The methods for sequencing a PMO described herein include contacting the crude digestion product with streptavidin-coated magnetic beads to obtain a magnetic bead isolate comprising free PMO and a magnetic bead complex comprising biotinylated PMO and streptavidin-coated magnetic beads. See FIGS. 2 and 3. The “contacting” can comprise mixing the streptavidin-coated magnetic beads with the crude digestion product in a suitable container. Streptavidin-coated magnetic beads are well known and are commercially available. One example of streptavidin-coated magnetic beads that can be used in the methods described herein are Dynabeads MyOne Streptavidin C1 beads available from ThermoFisher.

The magnetic bead isolate can be placed in a suitable container (an Eppendorf tube, such as the one shown in FIG. 3) and the mixture can be vortexed and placed on a magnetic stand for a time sufficient to allow the beads to accumulate. A supernatant can be removed (e.g., with a pipette or any suitable instrument) and the beads can be washed a suitable number of times by adding water and/or a suitable buffer (e.g., B&W buffer comprising 10 mM Tris-HCl, 1 mM EDTA, 2.0 M NaCl, pH=7.5) to the container following removal of the supernatant. Any free PMO is removed from the magnetic bead complex in this fashion to obtain a purified magnetic bead isolate.

The methods for sequencing a PMO described herein include releasing the streptavidin-coated magnetic beads from the magnetic bead complex to obtain a purified digestion product that can be analyzed by any suitable method or combinations of methods (e.g., chromatography-electrospray ionization-mass spectrometry (LC-ESI-MS), matrix assisted laser desorption ionization-time of flight mass spectrometry (MALDI-TOF MS), and nuclear magnetic resonance, and combinations thereof). The streptavidin-coated magnetic beads can be released from the magnetic bead complex in any suitable way, such as by heating the purified magnetic bead isolate at a temperature sufficient to release the purified acid digestion product from the streptavidin-coated magnetic beads. The temperature sufficient to release the purified acid digestion product from the streptavidin-coated magnetic beads can be at least 80° C. (e.g., at least 85° C., at least 90° C., at least 95° C., at least 100° C., from 80° C. to 100° C., 90° C. to 110° C. or 90° C. to 100° C.).

The purified digestion product will comprise digested PMOs of various different lengths as shown in FIG. 1. When analyzed by, e.g., MALDI-TOF, the spectrum shown in FIG. 2 is obtained. This spectrum is for the purified digestion product of PMO-1-PEG₂-biotin, wherein PMO-1 comprises the sequence is 5′ AAA CGC CGC CAT TTC TCA ACA GAT C 3′. The spectrum shows the mass of the PMOs of different lengths, wherein each letter represents a PMO that differs from the next by one nucleotide. Thus, for example, the peak at m/z 8256.384 represents the PMO-1 sequence minus a nucleotide comprising an adenine (A); the peak at m/z 7925.909 represents the PMO-1 sequence minus two nucleotides, each comprising an adenine; the peak at m/z 7595.050 represents the PMO-1 sequence minus three nucleotides, each comprising an adenine; and so on until the entire sequence is determined. The pattern shown in FIG. 2 is referred to herein as a “ladder-like deletion pattern.”

By “alkyl” and the prefix “alk” is meant a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic (e.g., C₃₋₂₄ cycloalkyl) or acyclic. The alkyl group can be branched or unbranched. The alkyl group can also be substituted or unsubstituted. For example, the alkyl group can be substituted with one, two, three or, in the case of alkyl groups of two carbons or more, four substituents independently selected from: (1) C₁₋₆ alkoxy (e.g., —O-Ak, wherein Ak is optionally substituted C₁₋₆ alkyl); (2) C₁₋₆ alkylsulfinyl (e.g., —S(O)-Ak, wherein Ak is optionally substituted C₁₋₆ alkyl); (3) C₁₋₆ alkylsulfonyl (e.g., —SO₂-Ak, wherein Ak is optionally substituted C₁₋₆ alkyl); (4) amino (e.g., —NR^{N1}R^{N2}, where each of R^{N1} and R^{N2} is, independently, H or optionally substituted alkyl, or R^{N1} and R^{N2}, taken together with the nitrogen atom to which each are attached, form a heterocyclyl group); (5) aryl; (6) arylalkoxy (e.g., —O-L-Ar, wherein L is a bivalent form of optionally substituted alkyl and Ar is optionally substituted aryl); (7) aryloyl (e.g., —C(O)—Ar, wherein Ar is optionally substituted aryl); (8) azido (e.g., —N₃); (9) cyano (e.g., —CN); (10) carboxyaldehyde (e.g., —C(O)H); (11) C₃₋₈ cycloalkyl (e.g., a monovalent saturated or unsaturated non-aromatic cyclic C₃₋₈ hydrocarbon group); (12) halo (e.g., F, Cl, Br, or I); (13) heterocyclyl (e.g., a 5-, 6- or 7-membered ring, unless otherwise specified, containing one, two, three, or four non-carbon heteroatoms, such as nitrogen, oxygen, phosphorous, sulfur, or halo); (14) heterocyclyloxy (e.g., —O-Het, wherein Het is heterocyclyl, as described herein); (15) heterocyclyloyl (e.g., —C(O)-Het, wherein Het is heterocyclyl, as described herein); (16) hydroxyl (e.g., —OH); (17) N-protected amino; (18) nitro (e.g., —NO₂); (19) oxo (e.g., ═O) or hydroxyimino (e.g., ═N—OH); (20) C₃₋₈ spirocyclyl (e.g., an alkylene or heteroalkylene diradical, both ends of which are bonded to the same carbon atom of the parent group); (21) C₁₋₆ thioalkoxy (e.g., —S-Ak, wherein Ak is optionally substituted C₁₋₆ alkyl); (22) thiol (e.g., —SH); (23) —CO₂R^{A}, where R^{A} is selected from (a) hydrogen, (b) C₁₋₆ alkyl, (c) C₄₋₁₈ aryl, and (d) (C₄₋₁₈ aryl) C₁₋₆ alkyl (e.g., -L-Ar, wherein L is a bivalent form of optionally substituted alkyl group and Ar is optionally substituted aryl); (24) —C(O)NR^{B}R^{D}, where each of R^{B} and R^{D} is, independently, selected from (a) hydrogen, (b) C₁₋₆ alkyl, (c) C₄₋₁₈ aryl, and (d) (C₄₋₁₈ aryl) C₁₋₆ alkyl (e.g., -L-Ar, wherein L is a bivalent form of optionally substituted alkyl group and Ar is optionally substituted aryl); (25) —SO₂R^{D}, where R^{D} is selected from (a) C₁₋₆ alkyl, (b) C₄₋₁₈ aryl, and (c) (C₄₋₁₈ aryl) C₁₋₆ alkyl (e.g., -L-Ar, wherein L is a bivalent form of optionally substituted alkyl group and Ar is optionally substituted aryl); (26) —SO₂NR^{E}R^{F}, where each of R^{E} and R^{F} is, independently, selected from (a) hydrogen, (b) C₁₋₆ alkyl, (c) C₄₋₁₈ aryl, and (d) (C₄₋₁₈ aryl) C₁₋₆ alkyl (e.g., -L-Ar, wherein L is a bivalent form of optionally substituted alkyl group and Ar is optionally substituted aryl); and (27) —NR^{G}R^{H}, where each of R^{G} and R^{H} is, independently, selected from (a) hydrogen, (b) an N-protecting group, (c) C₁₋₆ alkyl, (d) C₂₋₆ alkenyl (e.g., optionally substituted alkyl having one or more double bonds), (e) C₂₋₆ alkynyl (e.g., optionally substituted alkyl having one or more triple bonds), (f) C₄₋₁₈ aryl, (g) (C₄₋₁₈ aryl) C₁₋₆ alkyl (e.g., L-Ar, wherein L is a bivalent form of optionally substituted alkyl group and Ar is optionally substituted aryl), (h) C₃₋₈ cycloalkyl, and (i) (C₃₋₈ cycloalkyl) C₁₋₆ alkyl (e.g., -L-Cy, wherein L is a bivalent form of optionally substituted alkyl group and Cy is optionally substituted cycloalkyl, as described herein), wherein in one embodiment no two groups are bound to the nitrogen atom through a carbonyl group or a sulfonyl group. The alkyl group can be a primary, secondary, or tertiary alkyl group substituted with one or more substituents (e.g., one or more halo or alkoxy). In some embodiments, the unsubstituted alkyl group is a C₁₋₃, C₁₋₆, C₁₋₁₂, C₁₋₁₆, C₁₋₁₈, C₁₋₂₀, or C₁₋₂₄ alkyl group.

By “alkylene” is meant a multivalent (e.g., bivalent, trivalent, tetravalent, etc.) form of an alkyl group, as described herein. Exemplary alkylene groups include methylene, ethylene, propylene, butylene, etc. In some embodiments, the alkylene group is a C₁₋₃, C₁₋₆, C₁₋₁₂, C₁₋₁₆, C₁₋₁₈, C₁₋₂₀, or C₁₋₂₄, C₂₋₃, C₂₋₆, C₂₋₁₂, C₂₋₁₆, C₂₋₁₈, C₂₋₂₀, or C₂₋₂₄ alkylene group. The alkylene group can be branched or unbranched. The alkylene group can be saturated or unsaturated (e.g., having one or more double bonds or triple bonds). The alkylene group can also be substituted or unsubstituted. For example, the alkylene group can be substituted with one or more substitution groups, as described herein for alkyl. In one instance, a substituted alkylene group can include an optionally substituted hydroxyalkylene (e.g., an optionally substituted alkylene substituted with one or more hydroxyl groups), an optionally substituted haloalkylene (e.g., an optionally substituted alkylene substituted with one or more halo groups), and the like.

By “halo” is meant fluoro (F), chloro (Cl), bromo (Br), or iodo (I).

By “haloalkyl” is meant an alkyl group, as defined herein, substituted with one or more halo.

By “haloalkylene” is meant an alkylene group, as defined herein, substituted with one or more halo.

By “heteroalkylene” is meant an alkylene group containing one, two, three, or four non-carbon heteroatoms (e.g., independently selected from nitrogen, oxygen, phosphorous, sulfur, selenium, or halo). The heteroalkylene group can be saturated or unsaturated (e.g., having one or more double bonds or triple bonds). The heteroalkylene group can be substituted or unsubstituted. For example, the heteroalkylene group can be substituted with one or more substitution groups, as described herein for alkyl.

By “aryl” is meant a group that contains any carbon-based aromatic group including, but not limited to, phenyl, benzyl, anthracenyl, anthryl, benzocyclobutenyl, benzocyclooctenyl, biphenylyl, chrysenyl, dihydroindenyl, fluoranthenyl, indacenyl, indenyl, naphthyl, phenanthryl, phenoxybenzyl, picenyl, pyrenyl, terphenyl, and the like, including fused benzo-C₄₋₈ cycloalkyl radicals such as, for instance, indanyl, tetrahydronaphthyl, fluorenyl, and the like. The term aryl also includes “heteroaryl,” which is defined as a group that contains an aromatic group that has at least one heteroatom incorporated within the ring of the aromatic group. Examples of heteroatoms include, but are not limited to, nitrogen, oxygen, sulfur, and phosphorus. Likewise, the term non-heteroaryl, which is also included in the term aryl, defines a group that contains an aromatic group that does not contain a heteroatom. The aryl group can be substituted or unsubstituted. The aryl group can be substituted with one, two, three, four, or five substituents independently selected from (1) C₁₋₆ alkanoyl (e.g., —C(O)-Ak, wherein Ak is optionally substituted C₁₋₆ alkyl); (2) C₁₋₆ alkyl; (3) C₁₋₆ alkoxy (e.g., —O-Ak, wherein Ak is optionally substituted C₁₋₆ alkyl); (4) C₁₋₆ alkoxy-C₁₋₆ alkyl (e.g., -L-O-Ak, wherein L is a bivalent form of optionally substituted alkyl group and Ak is optionally substituted C₁₋₆ alkyl); (5) C₁₋₆ alkylsulfinyl (e.g., —S(O)-Ak, wherein Ak is optionally substituted C₁₋₆ alkyl); (6) C₁₋₆ alkylsulfinyl-C₁₋₆ alkyl (e.g., -L-S(O)-Ak, wherein L is a bivalent form of optionally substituted alkyl group and Ak is optionally substituted C₁₋₆ alkyl); (7) C₁₋₆ alkylsulfonyl (e.g., —SO₂-Ak, wherein Ak is optionally substituted C₁₋₆ alkyl); (8) C₁₋₆ alkylsulfonyl-C₁₋₆ alkyl (e.g., -L-SO₂-Ak, wherein L is a bivalent form of optionally substituted alkyl group and Ak is optionally substituted C₁₋₆ alkyl); (9) aryl; (10) amino (e.g., —NR^{N1}R^{N2}, where each of R^{N1} and R^{N2} is, independently, H or optionally substituted alkyl, or R^{N1} and R^{N2}, taken together with the nitrogen atom to which each are attached, form a heterocyclyl group); (11) C₁₋₆ aminoalkyl (e.g., an alkyl group substituted by one or more —NR^{N1}R^{N2} groups, as described herein); (12) heteroaryl (e.g., a subset of heterocyclyl groups (e.g., a 5-, 6- or 7-membered ring, unless otherwise specified, containing one, two, three, or four non-carbon heteroatoms), which are aromatic); (13) (C₄₋₁₈ aryl) C₁₋₆ alkyl (e.g., -L-Ar, wherein L is a bivalent form of optionally substituted alkyl and Ar is optionally substituted aryl); (14) aryloyl (e.g., —C(O)—Ar, wherein Ar is optionally substituted aryl); (15) azido (e.g., —N₃); (16) cyano (e.g., —CN); (17) C₁₋₆ azidoalkyl (e.g., an alkyl group substituted by one or more azido groups, as described herein); (18) carboxyaldehyde (e.g., —C(O)H); (19) carboxyaldehyde-C₁₋₆ alkyl (e.g., an alkyl group substituted by one or more carboxyaldehyde groups, as described herein); (20) C₃₋₈ cycloalkyl (e.g., a monovalent saturated or unsaturated non-aromatic cyclic C₃₋₈ hydrocarbon group); (21) (C₃₋₈ cycloalkyl) C₁₋₆ alkyl (e.g., an alkyl group substituted by one or more cycloalkyl groups, as described herein); (22) halo (e.g., F, Cl, Br, or I); (23) C₁₋₆ haloalkyl (e.g., an alkyl group substituted by one or more halo groups, as described herein); (24) heterocyclyl (e.g., a 5-, 6- or 7-membered ring, unless otherwise specified, containing one, two, three, or four non-carbon heteroatoms, such as nitrogen, oxygen, phosphorous, sulfur, or halo); (25) heterocyclyloxy (e.g., —O-Het, wherein Het is heterocyclyl, as described herein); (26) heterocyclyloyl (e.g., —C(O)-Het, wherein Het is heterocyclyl, as described herein); (27) hydroxyl (e.g., —OH); (28) C₁₋₆ hydroxyalkyl (e.g., an alkyl group substituted by one or more hydroxyl); (29) nitro (e.g., —NO₂); (30) C₁₋₆ nitroalkyl (e.g., an alkyl group substituted by one or more nitro, as described herein); (31) N-protected amino; (32) N-protected amino-C₁₋₆ alkyl (e.g., an alkyl group substituted by one or more N-protected amino groups); (33) oxo (e.g., ═O) or hydroxyimino (e.g., ═N—OH); (34) C₁₋₆ thioalkoxy (e.g., —S-Ak, wherein Ak is optionally substituted C₁₋₆ alkyl); (35) thio-C₁₋₆ alkoxy-C₁₋₆ alkyl (e.g., -L-S-Ak, wherein L is a bivalent form of optionally substituted alkyl and Ak is optionally substituted C₁₋₆ alkyl); (36) —(CH₂)ᵣCO₂R^{A}, where r is an integer of from zero to four, and R^{A} is selected from (a) hydrogen, (b) C₁₋₆ alkyl, (c) C₄₋₁₈ aryl, and (d) (C4 is aryl) C₁₋₆ alkyl (e.g., -L-Ar, wherein L is a bivalent form of optionally substituted alkyl and Ar is optionally substituted aryl); (37) —(CH₂)ᵣCONR^{B}R^{C}, where r is an integer of from zero to four and where each R^{B} and R^{D} is independently selected from (a) hydrogen, (b) C₁₋₆ alkyl, (c) C₄₋₁₈ aryl, and (d) (C₄₋₁₈ aryl) C₁₋₆ alkyl (e.g., -L-Ar, wherein L is a bivalent form of optionally substituted alkyl and Ar is optionally substituted aryl); (38) —(CH₂)ᵣSO₂R^{D}, where r is an integer of from zero to four and where R^{D} is selected from (a) C₁₋₆ alkyl, (b) C₄₋₁₈ aryl, and (c) (C₄₋₁₈ aryl) C₁₋₆ alkyl (e.g., -L-Ar, wherein L is a bivalent form of optionally substituted alkyl and Ar is optionally substituted aryl); (39) —(CH₂)ᵣSO₂NR^{E}R^{F}, where r is an integer of from zero to four and where each of R^{E} and R^{F} is, independently, selected from (a) hydrogen, (b) C₁₋₆ alkyl, (c) C₄₋₁₈ aryl, and (d) (C₄₋₁₈ aryl) C₁₋₆ alkyl (e.g., -L-Ar, wherein L is a bivalent form of optionally substituted alkyl and Ar is optionally substituted aryl); (40) —(CH₂)ᵣNR^{G}R^{H}, where r is an integer of from zero to four and where each of R^{G} and R^{H} is, independently, selected from (a) hydrogen, (b) an N-protecting group, (c) C₁₋₆ alkyl, (d) C₂₋₆ alkenyl (e.g., optionally substituted alkyl having one or more double bonds), (e) C₂₋₆ alkynyl (e.g., optionally substituted alkyl having one or more triple bonds), (f) C₄₋₁₈ aryl, (g) (C₄₋₁₈ aryl) C₁₋₆ alkyl (e.g., -L-Ar, wherein L is a bivalent form of optionally substituted alkyl and Ar is optionally substituted aryl), (h) C₃₋₈ cycloalkyl, and (i) (C₃₋₈ cycloalkyl) C₁₋₆ alkyl (e.g., -L-Cy, wherein L is a bivalent form of optionally substituted alkyl and Cy is optionally substituted cycloalkyl, as described herein), wherein in one embodiment no two groups are bound to the nitrogen atom through a carbonyl group or a sulfonyl group; (41) thiol (e.g., —SH); (42) perfluoroalkyl (e.g., an alkyl group having each hydrogen atom substituted with a fluorine atom); (43) perfluoroalkoxy (e.g., —OR^{F}, where R^{F} is an alkyl group having each hydrogen atom substituted with a fluorine atom); (44) aryloxy (e.g., —OAr, where Ar is optionally substituted aryl); (45) cycloalkoxy (e.g., —O-Cy, wherein Cy is optionally substituted cycloalkyl, as described herein); (46) cycloalkylalkoxy (e.g., —O-L-Cy, wherein L is a bivalent form of optionally substituted alkyl and Cy is optionally substituted cycloalkyl, as described herein); and (47) arylalkoxy (e.g., —O-L-Ar, wherein L is a bivalent form of optionally substituted alkyl and Ar is optionally substituted aryl). In particular embodiments, an unsubstituted aryl group is a C₄₋₁₈, C₄₋₁₄, C₄₋₁₂, C₄₋₁₀, C₆₋₁₈, C₆₋₁₄, C₆₋₁₂, or C₆₋₁₀ aryl group.

By “arylene” is meant a multivalent (e.g., bivalent, trivalent, tetravalent, etc.) form of an aryl group, as described herein. Exemplary arylene groups include phenylene, naphthylene, biphenylene, triphenylene, diphenyl ether, acenaphthenylene, anthrylene, or phenanthrylene. In some embodiments, the arylene group is a C₄₋₁₈, C₄₋₁₄, C₄₋₁₂, C₄₋₁₀, C₆₋₁₈, C₆₋₁₄, C₆₋₁₂, or C₆₋₁₀ arylene group. The arylene group can be branched or unbranched. The arylene group can also be substituted or unsubstituted. For example, the arylene group can be substituted with one or more substitution groups, as described herein for aryl.

By “heterocyclyl” is meant a 3-, 4-, 5-, 6- or 7-membered ring (e.g., a 5-, 6- or 7-membered ring), unless otherwise specified, containing one, two, three, or four non-carbon heteroatoms (e.g., independently selected from nitrogen, oxygen, phosphorous, sulfur, selenium, or halo). The 3-membered ring has zero to one double bonds, the 4- and 5-membered ring has zero to two double bonds, and the 6- and 7-membered rings have zero to three double bonds. The term “heterocyclyl” also includes bicyclic, tricyclic and tetracyclic groups in which any of the above heterocyclic rings is fused to one, two, or three rings independently selected from an aryl ring, a cyclohexane ring, a cyclohexene ring, a cyclopentane ring, a cyclopentene ring, and another monocyclic heterocyclic ring, such as indolyl, quinolyl, isoquinolyl, tetrahydroquinolyl, benzofuryl, benzothienyl and the like. Heterocyclics include acridinyl, adenyl, alloxazinyl, azaadamantanyl, azabenzimidazolyl, azabicyclononyl, azacycloheptyl, azacyclooctyl, azacyclononyl, azahypoxanthinyl, azaindazolyl, azaindolyl, azecinyl, azepanyl, azepinyl, azetidinyl, azetyl, aziridinyl, azirinyl, azocanyl, azocinyl, azonanyl, benzimidazolyl, benzisothiazolyl, benzisoxazolyl, benzodiazepinyl, benzodiazocinyl, benzodihydrofuryl, benzodioxepinyl, benzodioxinyl, benzodioxanyl, benzodioxocinyl, benzodioxolyl, benzodithiepinyl, benzodithiinyl, benzodioxocinyl, benzofuranyl, benzophenazinyl, benzopyranonyl, benzopyranyl, benzopyrenyl, benzopyronyl, benzoquinolinyl, benzoquinolizinyl, benzothiadiazepinyl, benzothiadiazolyl, benzothiazepinyl, benzothiazocinyl, benzothiazolyl, benzothienyl, benzothiophenyl, benzothiazinonyl, benzothiazinyl, benzothiopyranyl, benzothiopyronyl, benzotriazepinyl, benzotriazinonyl, benzotriazinyl, benzotriazolyl, benzoxathiinyl, benzotrioxepinyl, benzoxadiazepinyl, benzoxathiazepinyl, benzoxathiepinyl, benzoxathiocinyl, benzoxazepinyl, benzoxazinyl, benzoxazocinyl, benzoxazolinonyl, benzoxazolinyl, benzoxazolyl, benzylsultamyl benzylsultimyl, bipyrazinyl, bipyridinyl, carbazolyl (e.g., 4H-carbazolyl), carbolinyl (e.g., (3-carbolinyl), chromanonyl, chromanyl, chromenyl, cinnolinyl, coumarinyl, cytdinyl, cytosinyl, decahydroisoquinolinyl, decahydroquinolinyl, diazabicyclooctyl, diazetyl, diaziridinethionyl, diaziridinonyl, diaziridinyl, diazirinyl, dibenzisoquinolinyl, dibenzoacridinyl, dibenzocarbazolyl, dibenzofuranyl, dibenzophenazinyl, dibenzopyranonyl, dibenzopyronyl (xanthonyl), dibenzoquinoxalinyl, dibenzothiazepinyl, dibenzothiepinyl, dibenzothiophenyl, dibenzoxepinyl, dihydroazepinyl, dihydroazetyl, dihydrofuranyl, dihydrofuryl, dihydroisoquinolinyl, dihydropyranyl, dihydropyridinyl, dihydroypyridyl, dihydroquinolinyl, dihydrothienyl, dihydroindolyl, dioxanyl, dioxazinyl, dioxindolyl, dioxiranyl, dioxenyl, dioxinyl, dioxobenzofuranyl, dioxolyl, dioxotetrahydrofuranyl, dioxothiomorpholinyl, dithianyl, dithiazolyl, dithienyl, dithiinyl, furanyl, furazanyl, furoyl, furyl, guaninyl, homopiperazinyl, homopiperidinyl, hypoxanthinyl, hydantoinyl, imidazolidinyl, imidazolinyl, imidazolyl, indazolyl (e.g., 1H-indazolyl), indolenyl, indolinyl, indolizinyl, indolyl (e.g., 1H-indolyl or 3H-indolyl), isatinyl, isatyl, isobenzofuranyl, isochromanyl, isochromenyl, isoindazoyl, isoindolinyl, isoindolyl, isopyrazolonyl, isopyrazolyl, isoxazolidiniyl, isoxazolyl, isoquinolinyl, isoquinolinyl, isothiazolidinyl, isothiazolyl, morpholinyl, naphthindazolyl, naphthindolyl, naphthiridinyl, naphthopyranyl, naphthothiazolyl, naphthothioxolyl, naphthotriazolyl, naphthoxindolyl, naphthyridinyl, octahydroisoquinolinyl, oxabicycloheptyl, oxauracil, oxadiazolyl, oxazinyl, oxaziridinyl, oxazolidinyl, oxazolidonyl, oxazolinyl, oxazolonyl, oxazolyl, oxepanyl, oxetanonyl, oxetanyl, oxetyl, oxtenayl, oxindolyl, oxiranyl, oxobenzoisothiazolyl, oxochromenyl, oxoisoquinolinyl, oxoquinolinyl, oxothiolanyl, phenanthridinyl, phenanthrolinyl, phenazinyl, phenothiazinyl, phenothienyl (benzothiofuranyl), phenoxathiinyl, phenoxazinyl, phthalazinyl, phthalazonyl, phthalidyl, phthalimidinyl, piperazinyl, piperidinyl, piperidonyl (e.g., 4-piperidonyl), pteridinyl, purinyl, pyranyl, pyrazinyl, pyrazolidinyl, pyrazolinyl, pyrazolopyrimidinyl, pyrazolyl, pyridazinyl, pyridinyl, pyridopyrazinyl, pyridopyrimidinyl, pyridyl, pyrimidinyl, pyrimidyl, pyronyl, pyrrolidinyl, pyrrolidonyl (e.g., 2-pyrrolidonyl), pyrrolinyl, pyrrolizidinyl, pyrrolyl (e.g., 2H-pyrrolyl), pyrylium, quinazolinyl, quinolinyl, quinolizinyl (e.g., 4H-quinolizinyl), quinoxalinyl, quinuclidinyl, selenazinyl, selenazolyl, selenophenyl, succinimidyl, sulfolanyl, tetrahydrofuranyl, tetrahydrofuryl, tetrahydroisoquinolinyl, tetrahydroisoquinolyl, tetrahydropyridinyl, tetrahydropyridyl (piperidyl), tetrahydropyranyl, tetrahydropyronyl, tetrahydroquinolinyl, tetrahydroquinolyl, tetrahydrothienyl, tetrahydrothiophenyl, tetrazinyl, tetrazolyl, thiadiazinyl (e.g., 6H-1,2,5-thiadiazinyl or 2H,6H-1,5,2-dithiazinyl), thiadiazolyl, thianthrenyl, thianyl, thianaphthenyl, thiazepinyl, thiazinyl, thiazolidinedionyl, thiazolidinyl, thiazolyl, thienyl, thiepanyl, thiepinyl, thietanyl, thietyl, thiiranyl, thiocanyl, thiochromanonyl, thiochromanyl, thiochromenyl, thiodiazinyl, thiodiazolyl, thioindoxyl, thiomorpholinyl, thiophenyl, thiopyranyl, thiopyronyl, thiotriazolyl, thiourazolyl, thioxanyl, thioxolyl, thymidinyl, thyminyl, triazinyl, triazolyl, trithianyl, urazinyl, urazolyl, uretidinyl, uretinyl, uricyl, uridinyl, xanthenyl, xanthinyl, xanthionyl, and the like, as well as modified forms thereof (e.g., including one or more oxo and/or amino) and salts thereof. The heterocyclyl group can be substituted or unsubstituted. For example, the heterocyclyl group can be substituted with one or more substitution groups, as described herein for aryl.

By “heterocycle” is meant a compound having one or more heterocyclyl moieties. Non-limiting heterocycles include optionally substituted imidazole, optionally substituted triazole, optionally substituted tetrazole, optionally substituted pyrazole, optionally substituted imidazoline, optionally substituted pyrazoline, optionally substituted imidazolidine, optionally substituted pyrazolidine, optionally substituted pyrrole, optionally substituted pyrroline, optionally substituted pyrrolidine, optionally substituted tetrahydrofuran, optionally substituted furan, optionally substituted thiophene, optionally substituted oxazole, optionally substituted isoxazole, optionally substituted isothiazole, optionally substituted thiazole, optionally substituted oxathiolane, optionally substituted oxadiazole, optionally substituted thiadiazole, optionally substituted sulfolane, optionally substituted succinimide, optionally substituted thiazolidinedione, optionally substituted oxazolidone, optionally substituted hydantoin, optionally substituted pyridine, optionally substituted piperidine, optionally substituted pyridazine, optionally substituted piperazine, optionally substituted pyrimidine, optionally substituted pyrazine, optionally substituted triazine, optionally substituted pyran, optionally substituted pyrylium, optionally substituted tetrahydropyran, optionally substituted dioxine, optionally substituted dioxane, optionally substituted dithiane, optionally substituted trithiane, optionally substituted thiopyran, optionally substituted thiane, optionally substituted oxazine, optionally substituted morpholine, optionally substituted thiazine, optionally substituted thiomorpholine, optionally substituted cytosine, optionally substituted thymine, optionally substituted uracil, optionally substituted thiomorpholine dioxide, optionally substituted indene, optionally substituted indoline, optionally substituted indole, optionally substituted isoindole, optionally substituted indolizine, optionally substituted indazole, optionally substituted benzimidazole, optionally substituted azaindole, optionally substituted azaindazole, optionally substituted pyrazolopyrimidine, optionally substituted purine, optionally substituted benzofuran, optionally substituted isobenzofuran, optionally substituted benzothiophene, optionally substituted benzisoxazole, optionally substituted anthranil, optionally substituted benzisothiazole, optionally substituted benzoxazole, optionally substituted benzthiazole, optionally substituted benzthiadiazole, optionally substituted adenine, optionally substituted guanine, optionally substituted tetrahydroquinoline, optionally substituted dihydroquinoline, optionally substituted dihydroisoquinoline, optionally substituted quinoline, optionally substituted isoquinoline, optionally substituted quinolizine, optionally substituted quinoxaline, optionally substituted phthalazine, optionally substituted quinazoline, optionally substituted cinnoline, optionally substituted naphthyridine, optionally substituted pyridopyrimidine, optionally substituted pyridopyrazine, optionally substituted pteridine, optionally substituted chromene, optionally substituted isochromene, optionally substituted chromenone, optionally substituted benzoxazine, optionally substituted quinolinone, optionally substituted isoquinolinone, optionally substituted carbazole, optionally substituted dibenzofuran, optionally substituted acridine, optionally substituted phenazine, optionally substituted phenoxazine, optionally substituted phenothiazine, optionally substituted phenoxathiine, optionally substituted quinuclidine, optionally substituted azaadamantane, optionally substituted dihydroazepine, optionally substituted azepine, optionally substituted diazepine, optionally substituted oxepane, optionally substituted thiepine, optionally substituted thiazepine, optionally substituted azocane, optionally substituted azocine, optionally substituted thiocane, optionally substituted azonane, optionally substituted azecine, etc. Optional substitutions include any described herein for aryl. Heterocycles can also include cations and/or salts of any of these (e.g., any described herein, such as optionally substituted piperidinium, optionally substituted pyrrolidinium, optionally substituted pyrazolium, optionally substituted imidazolium, optionally substituted pyridinium, optionally substituted quinolinium, optionally substituted isoquinolinium, optionally substituted acridinium, optionally substituted phenanthridinium, optionally substituted pyridazinium, optionally substituted pyrimidinium, optionally substituted pyrazinium, optionally substituted phenazinium, or optionally substituted morpholinium).

Unless otherwise indicated, all numbers expressing quantities of components, molecular weights, and so forth used in the specification and claims are to be understood as being modified in all instances by the term “about.” As used herein in connection with a measured quantity, the term “about” refers to that variation in the measured quantity as would be expected by the skilled artisan making the measurement and exercising a level of care commensurate with the objective of the measurement and the precision of the measuring equipment used. Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Values expressed in a range format should be interpreted in a flexible manner to include not only the numerical values explicitly recited as the limits of the range, but also to include all the individual numerical values or sub-ranges encompassed within that range as if each numerical value and sub-range were explicitly recited. For example, a range of “0.1% to 5%” or “0.1% to 5%” should be interpreted to include not just as 0.1% to 5%, but also the individual values (for example, 1%, 2%, 3%, and 4%) and the sub-ranges (for example, 0.1% to 0.5%, 1.1% to 2.2%, 3.3% to 4.4%) within the indicated range. In addition, the term “in the range” or “within a range” (and similar statements) includes the endpoints of the stated range. Herein, for example, “up to” a number (for example, “up to 50”) includes the number (for example, 50).

In this document, the terms “a,” “an,” or “the” are used to include one or more than one unless the context clearly dictates otherwise. The term “or” is used to refer to a nonexclusive “or” unless otherwise indicated. Further, term “and/or” means one or all of the listed elements or a combination of any two or more of the listed elements.

In addition, it is to be understood that the phraseology or terminology employed herein, and not otherwise defined, is for the purpose of description only and not of limitation. Any use of section headings is intended to aid reading of the document and is not to be interpreted as limiting. Further, information that is relevant to a section heading may occur within or outside of that particular section. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

In the methods described herein, the steps may be carried out in any order without departing from the principles of the invention, except when a temporal or operational sequence is explicitly recited. Furthermore, specified steps may be carried out concurrently unless explicit claim language recites that they be carried out separately. For example, a claimed step of doing X and a claimed step of doing Y may be conducted simultaneously within a single operation, and the resulting process will fall within the literal scope of the claimed process.

The term “comprises” and variations thereof do not have a limiting meaning where these terms appear in the description and claims. Such terms will be understood to imply the inclusion of a stated step or element or group of steps or elements but not the exclusion of any other step or element or group of steps or elements.

By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

Unless otherwise specified, “a,” “an,” “the,” and “at least one” are used interchangeably and mean one or more than one.

Those skilled in the art will appreciate that many modifications to the embodiments described herein are possible without departing from the spirit and scope of the present disclosure. Thus, the description is not intended and should not be construed to be limited to the examples given but should be granted the full breadth of protection afforded by the appended claims and equivalents thereto. In addition, it is possible to use some of the features of the present disclosure without the corresponding use of other features. Accordingly, the foregoing description of or illustrative embodiments is provided for the purpose of illustrating the principles of the present disclosure and not in limitation thereof and may include modification thereto and permutations thereof.

### EXAMPLES

The disclosure can be better understood by reference to the following examples which are offered by way of illustration. The disclosure is not limited to the examples given herein.

### Example 1: Biotinylation Procedure

Biotin-PEG₂-OH (1 mg, BroadPharm) was dissolved in dimethyl sulfoxide (DMSO; 65 μL, Sigma-Aldrich), followed by addition of N,N-diisopropylethylamine; (DIPEA; 3 μL, Sigma-Aldrich) and 1-[Bis(dimethylamino)methylene]-1H-1,2,3-triazolo[4,5-b]pyridinium 3-oxide hexafluorophosphate (HATU, 12 μL of a freshly prepared 300 mM solution in DMSO) and the solution was stirred for 5 min at room temperature (rt.). This solution was added to a solution of PMO (ca. 8 mg dissolved in 100 μL of DMSO) and the mixture was stirred for 1 hour at rt. The progress of the reaction was monitored by liquid chromatography-electrospray ionization-mass spectrometry (LC-ESI-MS). If the starting material was not fully consumed, additional amounts of DIPEA and HATU were added until no more starting PMO could be detected by LC-ESI-MS. The duration of this coupling reaction turned out to be sequence dependent, with PMO-1 (5′ AAA CGC CGC CAT TTC TCA ACA GAT C 3′) fully consumed in less than an hour, whilst 3 days were required for full consumption of PMO-2 (5′ CAG CAG CAG CAG CAG CAG CAG 3′). As a competing reaction, acetylation of the secondary amine of the morpholine moiety in the PMO was detected by LC-ESI-MS and its relative amount was higher for slower reacting PMOs as it was accumulating over time. The reaction mixture was diluted with deionized (DI) water to a total volume of 1.5 mL and purified on a PD-10 size-exclusion cartridge (Cytiva). Before loading the sample, the cartridge was equilibrated by first washing with a buffer (20 mM HEPES, 130 mM NaCl) and then with DI water. The crude mixture of the biotinylated PMO was loaded on the cartridge and eluted with DI water. Fractions (ca. 1 mL) were collected and were analyzed by LC-ESI-MS. Fractions containing the desired product were combined and the concentration of the resulting solution was estimated by measuring its absorption (NanoDrop from Thermo Fisher).

### Example 2: Acidic Digestion

The concentration of the final solution was adjusted to ca. 50-100 μM either by concentrating under vacuum or by diluting it with DI water. After adjusting the stock solution to the desired concentration, 100 μL of it were diluted with 100 μL of a freshly prepared 0.3 M solution of TFA in DI water and incubated at 37° C. for 1 hour upon shaking. These conditions however might require adjustment, depending on the sequence of the biotinylated PMO. For instance, 30 min was enough to digest PMO-1-PEG₂-biotin but was not enough for PMO-2-PEG₂-biotin which possess a self-complimentary sequence and therefore prone to aggregation. The resulting solution was cooled to rt by placing on ice and 20 μL of NH₄OH solution (1:10 v/v in DI water) was added to neutralize the solution and the resulting solution was lyophilized giving a white powder comprising the crude digestion product from PMO-1-PEG₂-biotin or PMO-2-PEG₂-biotin.

### Example 3: Concentration Using Streptavidin-Coated Beads

Dynabeads MyOne Streptavidin C1 beads (200 μL in azide-containing buffer; Thermo Fisher) were added to an Eppendorf tube. The tube was vortexed for 2 min and then placed on a magnetic stand (Thermo Fisher) until beads accumulated on the wall of the tube. The azide-containing buffer was carefully removed using a pipette. B&W buffer (10 mM Tris-HCl, 1 mM EDTA, 2.0 M NaCl, pH=7.5) was added and the tube was vortexed for 2 min and then left on a magnetic stand until beads accumulated on the wall of the tube (repeated twice more). The lyophilized crude digestion product from PMO-1-PEG₂-biotin or PMO-2-PEG₂-biotin was added to the Eppendorf tube containing the washed beads and 200 μL of B&W buffer and the solution was incubated for 15 min at room temperature upon gently shaking. The tube was vortexed for 2 minutes and placed on a magnetic stand, until beads concentrated on the wall of the Eppendorf tube and the supernatant was carefully removed with a pipette and discarded. The remaining beads were vortexed and washed (vortexing and washing three times) with the B&W buffer as described above. After the third vortexing, the supernatant was removed and the beads were suspended in DI water (100 μL). The suspension was placed on the ThermoMixer (Eppendorf) for 5 min at 80° C. and then transferred onto a magnetic stand. After beads accumulated on the wall of the Eppendorf tube, the supernatant was carefully removed with a pipette and used for MS analysis. The supernatant comprises the purified digestion product of PMO-1-PEG₂-biotin or PMO-2-PEG₂-biotin.

### Example 4: MALDI-TOF Analysis

Different matrices were screened to select the one that produces the highest signal-to-noise ratio when mixed with PMO-1-PEG2-biotin resulting in the selection of 2,5-dihydroxybenzoic acid (30 mg in 1 mL of 7:3 acetonitrile/water+0.1% FA). This freshly prepared matrix (15 μL) was mixed with the solution containing PMO-1-PEG2-biotin (5 μL of ca. 50-100 μM solution in DI water), vortexed and deposited on a target frame (0.8 μL). The signal was acquired using the maximum signal intensity in the negative mode upon averaging multiple scans (data not shown). The resulting spectra had high signal-to-noise ratio allowing for an unambiguous assignment of very deletion down to m/z=1 kDa.

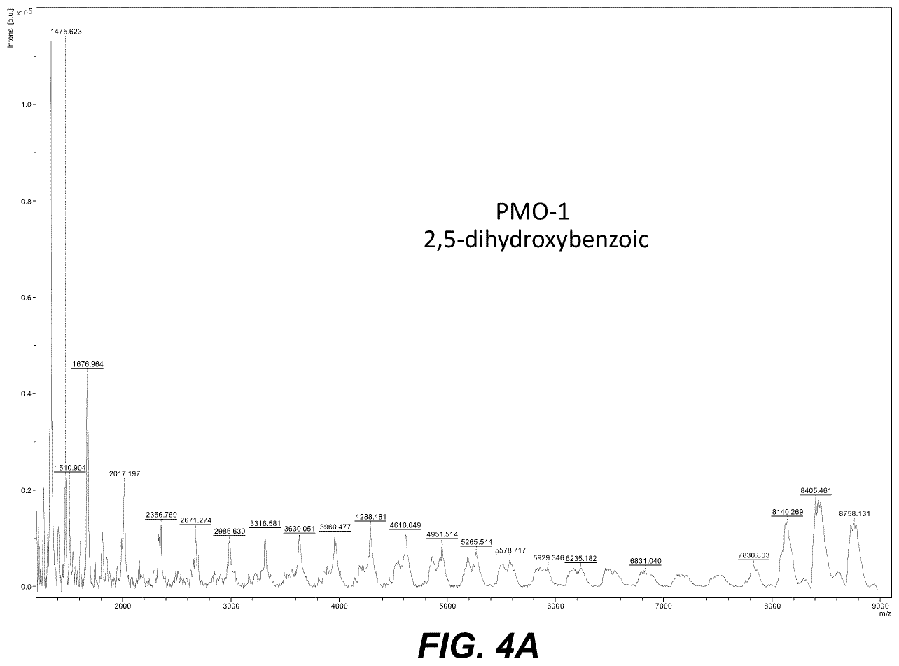

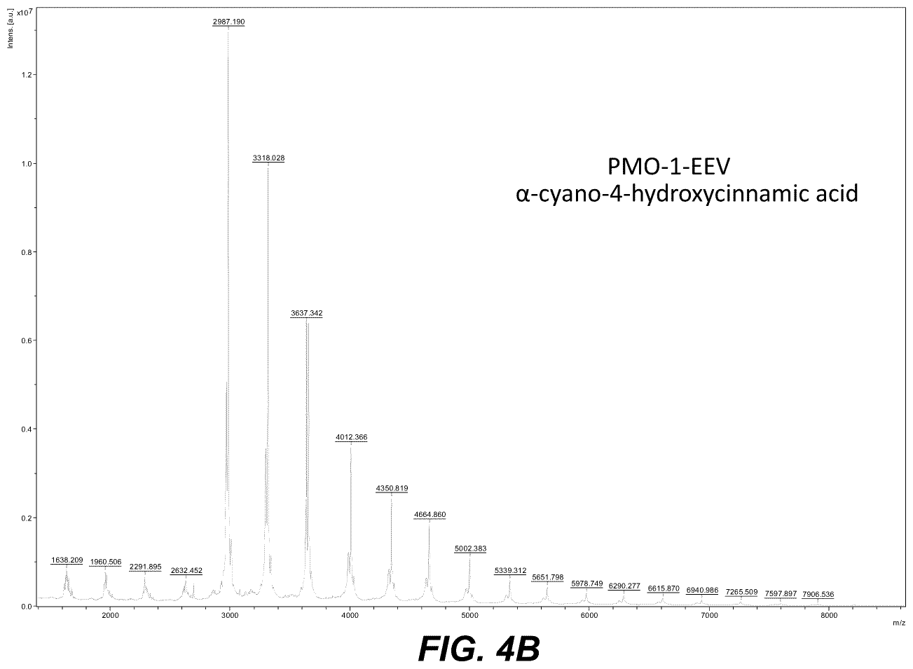

As a comparison, non-biotinylated PMOs were analyzed using the same matrix. Due to uncapped 5′- and 3′-ends, the acid-catalyzed truncation was happening from both ends, leading to spectra of significantly lower quality (FIG. 4A; PMO-1 using 2,5-dihydroxybenzoic acid as a matrix). At the same time, PPMOs had 3′-end coupled to a peptide, thereby producing spectra similar in quality to biotinylated PMOs (FIG. 4B; PPMO-1-EEV using α-cyano-4-hydroxycinnamic acid). In the latter case, α-cyano-4-hydroxycinnamic acid was used as a matrix as it produced a better crystallinity of the deposited sample.

What is claimed is:

1. A method for sequencing a phosphorodiamidate morpholino oligomer (PMO), the method comprising:
(a) conjugating a PMO to a biotin moiety to form a biotinylated PMO;
(b) contacting the biotinylated PMO with an acid to obtain a crude digestion product;
(c) contacting the crude digestion product with streptavidin-coated magnetic beads to obtain a magnetic bead isolate comprising free PMO and a magnetic bead complex comprising biotinylated PMO and streptavidin-coated magnetic beads;
(d) washing the magnetic bead isolate to remove free PMO from the magnetic bead complex to obtain a purified magnetic bead isolate;
(e) releasing the streptavidin-coated magnetic beads from the magnetic bead complex to obtain a purified digestion product; and
(f) analyzing the purified digestion product.

2. The method of claim 1, wherein acid is an inorganic acid.

3. The method of claim 1, wherein the acid is trifluoroacetic acid.

4. The method of claim 1, wherein the releasing the streptavidin-coated magnetic beads from the magnetic bead conjugate comprises heating the purified magnetic bead isolate at a temperature sufficient to release the purified acid digestion product from the streptavidin-coated magnetic beads.

5. The method of claim 1, wherein the PMO is a peptide-PMO (PPMO).

6. The method of claim 1, wherein the PMO comprises from 5 to 100 morpholino monomers.

7. The method of claim 1, wherein the biotin moiety is conjugated to the 3′ end of the PMO.

8. The method of claim 1, wherein the PMO is conjugated to the biotin moiety through a linker.

9. The method of claim 1, wherein the biotinylated PMO has the formula PMO-L-biotin, wherein L is a linker and the linker is covalently coupled to a 3′-end of the PMO.

10. The method of claim 1, wherein the PMO has a repeating sequence: 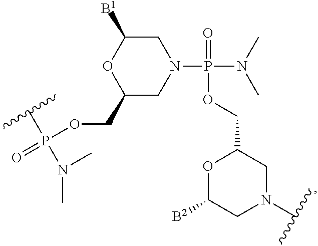
wherein B¹ and B² are each, independently, adenine, thymine, cytosine, guanine, uracil, or derivatives thereof.

11. The method of claim 9, wherein the PMO-L-biotin is of the formula: 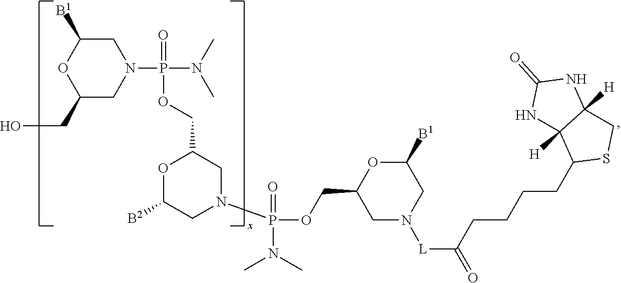
wherein
each B¹ and B² is, independently, adenine, thymine, cytosine, guanine, uracil, or derivatives thereof; and x is an integer from 10 to 100.

12. The method of claim 8, wherein the linker comprises a divalent polyethylene glycol (PEG) group.

13. The method of claim 8, wherein the linker comprises ionizable functional groups.

14. The method of claim 8, wherein the linker comprises or is a divalent group of the formula: 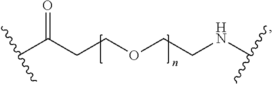
wherein n is an integer from 1 to 10.

15. The method of claim 8, wherein the linker comprises a divalent group of the formula: 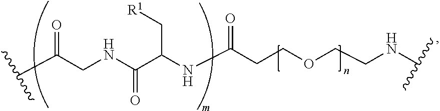
wherein:
R¹ comprises an ionizable group;
m is an integer from 1 to 10; and
n is an integer from 1 to 10.

16. The method of claim 8, wherein the ionizable group is an amino group or a guanidine group.

17. The method of claim 8, wherein the linker comprises a divalent group of the formula: 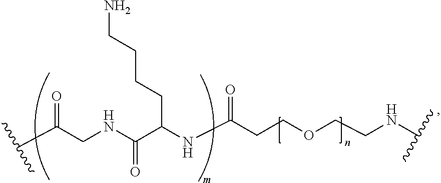
wherein:
m is an integer from 1 to 10; and
n is an integer from 1 to 10.

18. The method of claim 8, wherein the -L-biotin group comprises or is a group of the formula: 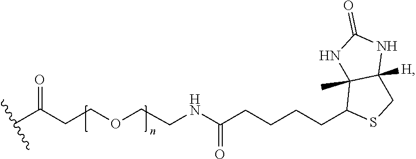
wherein n is an integer from 1 to 10.

19. The method of claim 14, wherein m is 2.

20. The method of claim 14, wherein n is 2.
